(19) United States
(12) Patent Application Publication
(10) Pub. No.: US 2017/0281311 A1
(43) Pub. Date: Oct. 5, 2017

(54) ARTICULATING ENDODONTIC FILE

(71) Applicants: Carlos Aloise, Alta Loma, CA (US); Ricardo Miramontes, Alta Loma, CA (US)

(72) Inventors: Carlos Aloise, Alta Loma, CA (US); Ricardo Miramontes, Alta Loma, CA (US)

(21) Appl. No.: 15/478,436

(22) Filed: Apr. 4, 2017

Related U.S. Application Data
(60) Provisional application No. 62/318,633, filed on Apr. 5, 2016.

(51) Int. Cl. A61C 5/42; A61B 17/16

(52) U.S. Cl. CPC A61C 5/42; A61B 17/1617

(57) ABSTRACT

Apparatus and methods for an articulating endodontic file are provided. The apparatus may include two or more file segments. Each file segment may articulate with respect to an adjacent file segment. The articulating file may include a joint between two file segments. The articulating file may include a first joint that allows a file segment to articulate in a first direction. The articulating file may include a second joint that allows a second file segment to articulate in a second direction. The articulating file may be configured such that torque applied to a first file segment is transmitted to one or more other file segments in the file.

### CROSS REFERENCE TO RELATED APPLICATION

This application is a nonprovisional of U.S. Provisional Patent Application No. 62/318,633 filed on Apr. 5, 2016, which is hereby incorporated herein by reference in its entirety.

### FIELD OF TECHNOLOGY

The present disclosure relates to the field of endodontic instrumentation. Specifically, the present disclosure relates to endodontic files used to clean, remove debris from, and/or shape a tooth's root canal during a dental procedure.

### BACKGROUND

A tooth may develop a carious lesion. The carious lesion may infect tooth tissue. A carious lesion may infect tooth tissue in a root canal of the tooth. If tooth tissue in the root canal is infected, the infected tissue may be removed from the tooth to stop further spreading of the carious lesion.

Shaping and cleaning a root canal of a tooth is often achieved by the use of endodontic files. The files typically have cutting edges for removing tissue in and/or near the root canal. The cutting edges are typically formed by fluting the file. The flutes typically extend helically along a length of the file. Root canals are seldom straight, and often include bends and twists. At least some endodontic files are preferably flexible.

An endodontic file should, preferably, when operating in the canal, preserve a natural curvature of the root canal. Preserving the natural curvature may include keeping an apical foramen as small as practical. A file should preferably shape the canal with no or minimal foramen transportation. Preserving the natural curvature may include minimizing any widening of the root canal. A file should preferably shape the canal without perforating the canal.

A root canal's shape differs from patient to patient. Typically, a root canal is narrow and tortuous. An endodontic practitioner may attempt to remove infected tissue from the root canal while minimizing damage to uninfected tooth tissue. This may be difficult, however, because typically files have a tendency to advance in a straight line along the file's longitudinal axis and may not be easily manipulated to follow the root canal's unique curvature.

It would be desirable, therefore, to provide an articulating endodontic file.

### BRIEF DESCRIPTION OF THE DRAWINGS

The objects and advantages of the disclosure will be apparent upon consideration of the following detailed description, taken in conjunction with the accompanying drawings, in which like reference characters refer to like parts throughout, and in which:

FIG. 1 shows illustrative apparatus in accordance with principles of the disclosure;

FIG. 2 shows illustrative apparatus in accordance with principles of the disclosure;

FIG. 3 shows illustrative apparatus in accordance with principles of the disclosure;

FIG. 4 shows illustrative apparatus in accordance with principles of the disclosure;

FIG. 4A shows illustrative apparatus in accordance with principles of the disclosure;

FIG. 5 shows illustrative apparatus in accordance with principles of the disclosure;

FIG. 6 shows illustrative apparatus in accordance with principles of the disclosure;

FIG. 7 shows illustrative apparatus in accordance with principles of the disclosure;

FIG. 8 shows illustrative apparatus in accordance with principles of the disclosure;

FIG. 9 shows illustrative apparatus in accordance with principles of the disclosure;

FIG. 10 shows illustrative apparatus in accordance with principles of the disclosure;

FIG. 11 shows illustrative apparatus in accordance with principles of the disclosure;

FIG. 12 shows illustrative apparatus in accordance with principles of the disclosure;

FIG. 13 shows illustrative apparatus in accordance with principles of the disclosure;

FIG. 14 shows illustrative apparatus in accordance with principles of the disclosure;

FIG. 15 shows illustrative apparatus in accordance with principles of the disclosure;

FIG. 16 shows illustrative apparatus in accordance with principles of the disclosure;

FIG. 17 shows illustrative apparatus in accordance with principles of the disclosure;

FIG. 18 shows illustrative apparatus in accordance with principles of the disclosure;

FIG. 19 shows illustrative apparatus in accordance with principles of the disclosure;

FIG. 20 shows illustrative apparatus in accordance with principles of the disclosure;

FIG. 21 shows illustrative apparatus in accordance with principles of the disclosure;

FIG. 22 shows illustrative apparatus in accordance with principles of the disclosure;

FIG. 23 shows illustrative apparatus in accordance with principles of the disclosure;

FIG. 24 shows illustrative apparatus in accordance with principles of the disclosure;

FIG. 25 shows illustrative apparatus in accordance with principles of the disclosure;

FIG. 26 shows illustrative apparatus in accordance with principles of the disclosure;

FIG. 27 shows illustrative apparatus in accordance with principles of the disclosure;

FIG. 28 shows illustrative apparatus in accordance with principles of the disclosure;

FIG. 29 shows illustrative apparatus that may be utilized in accordance with principles of the disclosure;

FIG. 30 shows illustrative apparatus that may be utilized in accordance with principles of the disclosure;

FIG. 31 shows illustrative apparatus that may be utilized in accordance with principles of the disclosure;

FIG. 32 shows illustrative apparatus that may be utilized in in accordance with principles of the disclosure;

FIG. 33 shows illustrative apparatus that may be utilized in accordance with principles of the disclosure;

FIG. 34 shows illustrative apparatus that may be utilized in accordance with principles of the disclosure;

FIG. 35 shows illustrative apparatus that may be utilized in accordance with principles of the disclosure;

FIG. 36 shows illustrative apparatus that may be utilized in accordance with principles of the disclosure;

FIG. 37 shows illustrative apparatus that may be utilized in accordance with principles of the disclosure;

FIG. 38 shows illustrative apparatus that may be utilized in accordance with principles of the disclosure;

FIG. 39 shows illustrative apparatus that may be utilized in accordance with principles of the disclosure; and

FIG. 40 shows illustrative apparatus that may be utilized in accordance with principles of the disclosure.

### DETAILED DESCRIPTION OF THE DISCLOSURE

Apparatus and methods for an articulating endodontic file are provided. The file may include one or more file segments. A first segment may be configured to move, bend, deflect, rotate and/or pivot relative to a second file segment. An articulating file may enhance the file's ability to follow a curvature of a patient's root canal during an endodontic procedure and minimize damage to uninfected tooth tissue.

The file may be a cannulated file as disclosed in U.S. patent application Ser. No. 15/367,376, which is hereby incorporated by reference herein in its entirety. A cannulated file may aid in channeling debris out of the root canal.

The articulating file may include a central member. The central member may extend through one or more of the file segments. The central member may have any suitable cross-section. For example the central member may have a circular cross-section or a cross-section having one or more sides, edges, protrusions and/or notches. The central member may be flexible. The central member may not have a uniform diameter along its length.

The central member may be rotated. The central member may rotated by a motor. The articulating file may include one or more file segments. The file segments may be cannulated. The central member may pass through one or more of the file segments. The central member may drive, in rotation, one or more of the file segments through which it passes. A file segment may be longitudinally fixed to the central member. A file segment may be slidable along the central member. A file segment may be removable from the central member. A file segment may not be removable from the central member.

The articulating file may include a joint. The joint may be positioned between a first file segment and a second file segment. The first file segment and/or the second file segment may deflect and/or pivot about the joint. A file segment may pivot at the joint relative to one or two adjacent file segments. A file segment may pivot at the joint relative to a longitudinal axis defined by one or two adjacent file segments. Illustrative joints may include a ball-and-socket, hinge, pivot or any other suitable joint.

The articulating file may include one or more spacers. Each spacer may be positioned between two file segments. The spacer may be rigid. The spacer may be flexible. For example, the spacer may be compressible. The spacer may compress in response to stress applied to the file or file segments. When a first segment deflects relative to a second segment, the spacer may be compressed. The spacer may limit movement of a file segment.

A spacer may provide an articulating file with a default shape. The spacer may provide the articulating file with a straight shape in an unstressed state. The spacer may provide the articulating file with a curved shape in an unstressed state. A file may include two or more spacers that may each have different flexibilities. Flexibility of a spacer may or may not determine the flexibility, or inflexibility, of a joint between two file segments.

A spacer may transmit force from one file segment to another file segment. The space may join one file segment to another file segment.

File segments may be formed from one or more of stainless steel and nickel-titanium (Ni—Ti) alloys. Such materials, particularly Ni—Ti alloys (e.g., copper nitinol and nickel nitinol), typically exhibit good flexibility, resilience and strength. Ni—Ti alloys also exhibit superelasticity and shape memory (or controlled memory) properties. Flexibility and strength reduce a likelihood of file or segment breakage when operating in the canal.

Apparatus for an articulating endodontic file is provided. The file includes a first file segment. The file includes a handle. The handle may define a proximal end of the file. The file includes a tip. The tip may define a distal end of the file.

The first file segment defines a longitudinal axis. The first file segment includes a first rib.

The file includes a second file segment. The second file segment may be positioned adjacent to the first file segment. The second file segment includes a second rib.

In operation, when a force is applied to the first file segment, the first rib engages the second rib. Engagement of the first rib and the second rib transmits the force from the first file segment to the second file segment. The first file segment transmits force to the second file segment when at least one rib in the plurality of first ribs interlocks with at least one rib in the plurality of second ribs.

In operation, the second file segment is configured to deflect relative to the longitudinal axis of the first segment. The second segment may deflect while receiving the force. The force may be torque or any suitable force.

The first rib may be one of a plurality of first ribs. The plurality of first ribs protrudes from a distal end of the first file segment. The second rib may be one of a plurality of second ribs. The plurality of second ribs protrudes from a proximal end of the second file segment.

Each of the plurality of first ribs is spaced about the distal end of the first file segment. The plurality of first ribs may be circumferentially distributed about a distal end of the first file segment. For example, the plurality of first ribs may be spaced about the distal end of the first file segment such that each rib in the plurality of second ribs is configured to fit between two adjacent ribs in the plurality of first ribs.

The articulating file may include a central member. The central member joins the first file segment to the second file segment. The first file segment may be affixed at a first position along the central member. The second file segment may be affixed to a second position along the central member. A file segment may be affixed longitudinally and/or rotationally at a position along the central axis.

The central member may pass through the first file segment. The central member may pass through the second file segment. The central member may have a non-circular cross-section. For example, the central member may have a triangular or square-shaped cross-section. One or more file segments may be cannulated. A cannula within a file segment may have a circular or non-circular cross-section. The central member may fit within the cannulated file segment and transmit force to the file segment mounted on the central member.

The first file segment may include a tapered surface. The tapered surface may be at a distal or proximal end of the file segment. In operation, at least one rib in the plurality of second ribs contacts the tapered surface of the first file segment when the second file segment deflects relative to the longitudinal axis of the first file segment.

In operation, when the second file segment deflects relative to the longitudinal axis of the first file segment, at least one rib in the plurality of second ribs interlocks with at least one rib in the plurality of first ribs. The interlocking of the at least one first rib and the at least one second rib allows force to be transmitted from the first file segment to the second file segment.

An articulating endodontic file may include a joint. The joint may be positioned between a distal end of the first file segment and a proximal end of the second file segment. The joint may include a distal end of the first file segment and a proximal end of the second file segment.

The joint allows the second file segment to articulate with respect to the first file segment. For example, in operation, the second file segment is configured to deflect relative to a longitudinal axis of the first file segment by bending about the joint. The joint provides the second file segment a range of motion relative to the first file segment. The range of motion may be 360 degrees about a longitudinal axis of the first file segment. The range of motion may be between 0 and 90 degrees in any direction about, and/or relative to, the longitudinal axis of the first segment.

The first file segment may be configured to deflect with respect to a longitudinal axis defined by the second file segment.

The first file segment may include at least one cutting edge. The second file segment may include at least one cutting edge. The cutting edge of the second file segment may be contiguous with the cutting edge of the first file segment. The cutting edges may be continuous when a longitudinal axis of the second file segment is aligned with a longitudinal axis of the first file segment.

Apparatus for an articulating endodontic file is provided. The file includes a first file segment. The first file segment includes a head. The head may be mounted on a neck. The neck may extend from a distal end of the file segment. The neck may extend from a proximal end of the file segment. The head may be spherically shaped. The head may include an articulating surface.

The file includes a second file segment. The file second segment includes a recess. The recess is configured to receive the head. The head may be “snap-fit” into the recess. The head is configured to articulate against a surface of the recess. The surface may form at least part of a distal end of a file segment.

The file includes a spacer. The spacer may be positioned between the first file segment and the second file segment. The spacer may be positioned between a distal end of a first file segment and a proximal end of a second file segment. The spacer may surround a file segment's neck.

The file may include a central member. The central member may join the first file segment to the second file segment. The first segment may include a first inner lumen. The second segment may include a second inner lumen. An inner lumen may extend through a head and/or recess of a file segment. For example, the first inner lumen may extend through the head of the first segment. The second inner lumen may extend through the recess of the second file segment. The central member extends through the first inner lumen and the second inner lumen.

The first inner lumen may have a non-circular cross-section. The central member may include a cross-section that is configured to engage the non-circular cross-section of the inner lumen. In operation, the central member engages the cross-section of an inner lumen such that force applied to the central member is transmitted to a file segment mounted on the central member. The force applied to the central member may be a twisting force such as torque.

An articulating file may include three or more file segments. Each file segment may include an inner lumen. The central member may pass through the inner lumen of each file segment. The central member may thereby join three or more file segments.

The first file segment has a length. The first file segment may be tapered along its length. The second file segment has a length. The second file segment may be tapered along its length. The length of the first file segment may be different from the length of the second file segment. The taper of the first file segment may be different from the taper of the second file segment. Illustrative tapers may include 02, 04, 06, 08, 100 and/or 120 size tapers.

A file segment may be fluted. Each file segment in an articulating file may be fluted. A flute may define one or more cutting edges.

Apparatus for an articulating endodontic file is provided. The file includes a central member. The file includes a first file segment mounted on the central member. The file includes a second file segment mounted on the central member.

The first file segment includes a first inner lumen. The second file segment includes a second inner lumen. The central member may include a non-circular cross-section. The non-circular cross-section may include one or more edges. The central member is configured to pass through the first inner lumen and pass through the second inner lumen. The inner lumens may define cross-sections that are configured to engage the one or more edges of the central member.

A force may be applied to the central member. The force may be applied to the central member by a motor. The force may be a twisting force such as torque. The central member is configured to transmit the force to the first file segment and/or to the second file segment. The central member transmits the force to the first file segment and to the second file segment when the one or more edges of the central member engage corresponding features of the first and second inner lumens.

The articulating file includes a spacer between the first file segment and the second file segment. A spacer may prevent debris from gathering (during a dental procedure) in a space between the first file segment and the second file segment.

The first file segment may include a distal recess. The distal recess is configured to receive a proximal end of the spacer. The second file segment may include a proximal recess. The proximal recess is configured to receive a distal end of the spacer.

The first file segment may include a first finger. The first finger extends from a distal end of the first file segment. The second file segment may include a second finger. The second finger extends from a proximal end of the second file segment.

A spacer may include at least two protrusions. The two protrusions define a notch. The first finger or the second finger is configured to be seated in the notch. The first finger or second finger may be seated snugly in the notch. “Seated snugly” includes being seated in the notch without any space between a finger and a protrusion.

Apparatus for an articulating endodontic file is provided. The file includes a first file segment. The file includes a second file segment. The file includes a joint linking the first file segment to the second file segment.

In operation, the second file segment articulates about the joint. The second file segment may articulate about the joint in a plane that is perpendicular, or substantially perpendicular, to a longitudinal axis of the first file segment.

The joint includes a proximal extension. The proximal extension is affixed to a proximal end of the second file segment. The joint includes a distal cutout in a distal end of the first file segment. The distal cutout may be “U” shaped. The distal cutout defines a first leg and a second leg in the first file segment. The distal cutout is configured to receive the proximal extension.

The joint includes a pin. The pin passes through the first leg (first file segment), through the proximal extension (second file segment) and through the second leg (second file segment). The pin joins the proximal extension of the second file segment to the legs of the first file segment thereby rotatably affixing the first file segment to the second file segment. The joint allows the first segment and the second segment to rotate about the pin. The pin may be positioned perpendicular, or substantially perpendicular to a longitudinal axis defined by the first segment.

The joint may be a first joint. The plane may be a first plane. The file may include a second joint. The file may include a third file segment. The second joint links the second file segment to the third file segment. In operation, the third file segment articulates about the second joint in a second plane. The second plane is perpendicular, or substantially perpendicular, to the longitudinal axis of the first file segment and perpendicular, or substantially perpendicular, to the first plane.

The second joint includes a proximal extension affixed to a proximal end of the third file segment. The second joint includes a pin. The pin of the second joint passes through the second file segment. The pin of the second joint may be positioned perpendicular, or substantially perpendicular, to the pin of the first joint. The pin may be affixed to the proximal extension of the third file segment. The pin may pass through the proximal extension of the third file segment. Engagement of the pin with the second file segment and with the proximal extension (of the third file segment) rotatably affixes the third file segment to the second file segment.

An articulating endodontic file may include any suitable number of first joints and/or second joints. An articulating endodontic file may include any suitable combination of first joints and/or second joints.

Apparatus and methods described herein are illustrative. Methods steps may be performed in an order other than the order shown and/or described herein. Some embodiments may omit steps shown and/or described in connection with the illustrative methods. Some embodiments may include steps that are neither shown nor described in connection with the illustrative methods. Illustrative method steps may be combined. For example, an illustrative method may include steps shown in connection with another illustrative method.

Apparatus may omit features shown and/or described in connection with illustrative apparatus. Some embodiments may include features that are neither shown nor described in connection with the illustrative methods. Features of illustrative apparatus may be combined. For example, one illustrative embodiment may include features shown in connection with another illustrative embodiment.

Apparatus may involve some or all of the features of the illustrative apparatus and/or some or all of the steps of the illustrative methods. Methods may involve some or all of the steps of the illustrative methods and/or some or all of the features of the illustrative apparatus.

Apparatus and methods will now be described with reference to the accompanying figures, which form a part hereof. It is to be understood that other embodiments may be utilized and that structural, functional and procedural modifications may be made without departing from the scope and spirit of the present disclosure.

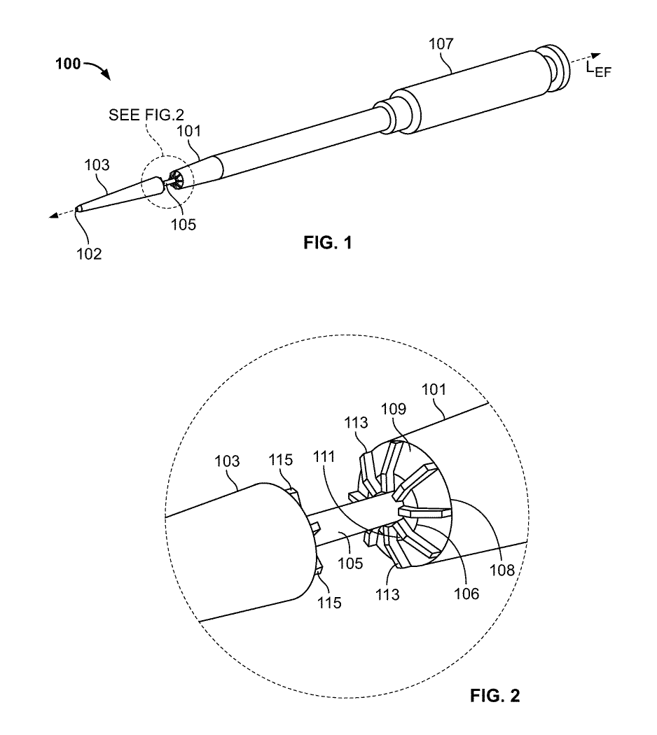

FIG. 1 shows exemplary endodontic file 100. File 100 may define a proximal end, a distal end, and longitudinal axis L_{EF}. For the purposes of this application, “proximal” and “distal” may be defined relative to a practitioner handling file 100. Thus, handle 107 corresponds to a proximal end and tip 102 corresponds to a distal end.

File 100 may have one or more beveled surfaces (not shown) designed for cutting tissue. For example, file 100 may be fluted. File 100 may have one or more cutting edges. File 100 may include one or more sides, flutes, grooves, channels, ribs and/or ridges.

File 100 may define any suitable cross section. A cross section may be circular, or may include any suitable number of sides, edges or vertices.

File 100 includes segment 101 and segment 103. In other embodiments, file 100 may include three or more file segments (not shown). FIG. 1 shows that segments 101 and 103 are tapered.

File 100 may include central member 105. Central member 105 may be flexible. Central member 105 may be substantially rigid. Central member 105 may be partially rigid. Central member 105 may have any suitable degree of flexibility. Central member 105 may include a degree of flexibility that varies along a length of central member 105.

In FIG. 1, central member 105 is illustrated as having a circular cross-section. In other embodiments, central member 105 may have any suitable cross-section such as a triangular cross-section, a rectangular cross-section, or any other suitable non-circular cross-section. In some of these embodiments, one or more edges of central member 105 may be used to transmit rotational motion from central member 105 to one or both of segment 101 and segment 103.

Central member 105 may traverse some or all of file 100. In some embodiments, central member 105 may be positioned along longitudinal axis L_{EF}. In some embodiments, central member 105 may be spaced apart from and parallel to (or anti-parallel to) longitudinal axis L_{EF}.

Central member 105 may be fixedly attached to at least a portion of file 100. For example, in some embodiments, a proximal end of central member 105 may be fixedly or removably attached to handle 107 of file 100. In some of these embodiments, a distal end of central member 105 may be fixedly or removably attached to segment 103. For example, a file segment may be crimped onto central member 105.

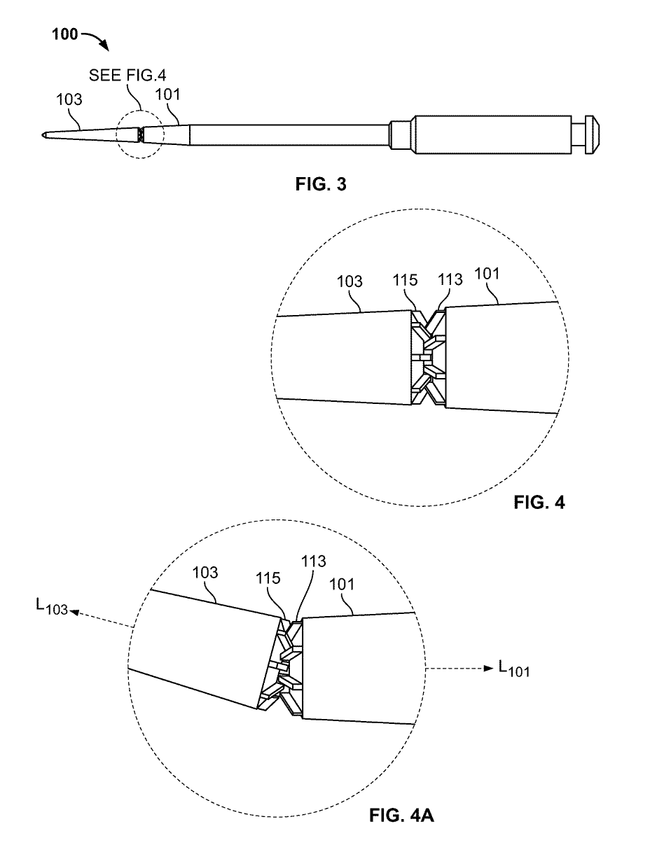

FIGS. 1-2 illustratively show segment 101 spaced apart from segment 103. FIGS. 1-2 also show central member 105 extending between segment 101 and segment 103. The spacing between segment 101 and segment 103 is shown for illustrative purposes only. In some embodiments, file 100 may have a contracted configuration (shown below in FIGS. 3-4A).

FIG. 2 shows an enlarged view of a circled area shown in FIG. 1.

FIG. 2 shows a distal end of segment 101. The distal end includes tapered surface 109. The distal end includes face 111. Face 111 may be circular. Face 111 may have any suitable shape. Segment 101 includes rib 113. Segment 101 includes a plurality of ribs 113. A rib, such as rib 113, may protrude from tapered surface 109. Rib 113 may protrude from face 111.

In some embodiments, rib 113 may extend along circular face 111 in a direction perpendicular to a central axis of central member 105. In some embodiments, rib 113 may extend along tapered surface 109 in a substantially straight path between outer circumference 108 of tapered surface 109 and inner circumference 106 of tapered surface 109. Inner circumference 106 also defines an outer perimeter of face 111.

Segment 103 includes a proximal end. The proximal end of segment 103 includes a tapered surface (not shown). The tapered surface of segment 103 may include one or more features of tapered surface 109. The proximal end of segment 103 includes a face (not shown). The face of segment 103 may include one or more features of face 111.

The proximal end of segment 103 includes rib 115. In some embodiments, the proximal end may include a plurality of ribs 115. In some embodiments, rib 115 may extend along a circular face of segment 103 in a direction perpendicular to a central axis of segment 103. In some embodiments, a rib 115 may extend along a tapered surface in a substantially straight path between an outer circumference and an inner circumference of the tapered surface.

Ribs 113 may be circumferentially distributed around tapered surface 109 and/or face 111. A first rib 113 may be adjacent to a second rib 113. The first rib 113 may be spaced apart from the second rib 113. A space between the first rib 113 and the second rib 113 may define an opening. The opening may be sized to receive rib 115 on adjacent segment 103. For example, the space between two ribs 113 may be sized to receive rib 115.

FIG. 3 shows another view of exemplary file 100. In FIG. 3, segment 101 is positioned along central member 105 (shown in FIGS. 1 and 2) such that a distal end of segment 101 abuts a proximal end of segment 103.

FIG. 4 shows an enlarged view of the circled area shown in FIG. 3. FIG. 4 shows a distal end of segment 101 abutting a proximal end of segment 103. In such a configuration, ribs 115 may be in physical contact with circular face 111 of segment 101. Each of ribs 115 may be positioned on circular face 111 between a first rib 113 and a second rib 113.

Each of ribs 113 may be positioned between a first rib 115 and a second rib 115. Ribs 113 may be in physical contact with segment 103. For example, ribs 113 may be in contact with a circular face (not shown) of segment 103.

Each of ribs 115 may physically contact one or two ribs 113. The physical contact may include a rib 115 interlocking with a rib 113. A rib 115 may interlock with a rib 113 when segment 101 or segment 103 is rotated. Interlocking contact between ribs 113 and ribs 115 may transmit a rotational force from segment 101 to segment 103 or vice versa. In some embodiments, multiple ribs 113 may interlock with multiple ribs 115.

File 100 may have one or more flexible properties. For example, in some embodiments, central member 105 may be flexible. Segments 101 and 103 may be constructed from material having shape memory or superelastic properties.

Segment 101 may not be rigidly attached to segment 103. For example, when operating in the canal, if a segment encounters tissue having a higher density relative to other tissue in the canal, the segment may deflect away from the longitudinal axis defined by file 100. If a segment encounters tissue having a higher density relative to a root canal, the segment may deflect away from the longitudinal axis defined by an adjacent segment. The segment may deflect away from the adjacent segment by bending about a joint between two segments.

A joint may include ribs 113 and 115. A joint may include one or more surfaces such as circular face 111 or tapered surface 109. Considering file 100, when segment 103 deflects away from longitudinal file axis L_{EF}, one or more of ribs 115 may abut tapered surface 109.

FIG. 4A shows file segment 103 deflecting relative to file segment 101. FIG. 4A shows that segment 101 defines longitudinal axis L₁₀₁. FIG. 4A shows that segment 103 defines longitudinal axis L₁₀₃. FIG. 4A shows that segment 103 may deflect relative to L₁₀₁. FIG. 4A shows that segment 101 may deflect relative to L₁₀₃.

Articulating and flexible properties of file 100 may improve the ability of file 100 to follow a natural curvature of a tooth's root canal. Articulating and flexible properties of file 100 may reduce a tendency of file 100 to advance through the canal in a straight line. Density differences in tissue within the root canal and surrounding the canal may guide deflection of segment 103 relative to segment 101.

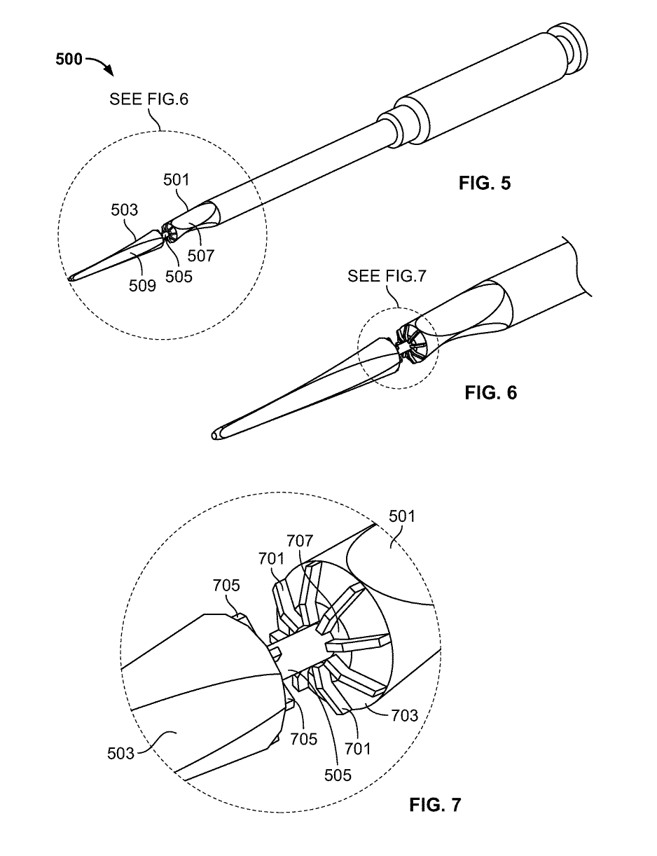

FIG. 5 shows illustrative articulating file 500. File 500 includes file segment 501, file segment 503, and central member 505. File 500 may have one or more properties in common with file 100 (shown above in FIG. 1).

File 500 includes flutes. The flutes include flute 507 and flute 509. The flutes enable file 500 to cut dental tissue. Geometric parameters of a flute may provide file 500 with specific cutting properties.

FIGS. 5-7 show segment 501 spaced apart from segment 503. FIGS. 5-7 also show central member 505 extending between segment 501 and segment 503. The spacing between segment 501 and segment 503 is shown for illustrative purposes only. In some embodiments, file 500 may be used in a contracted configuration (not shown). The contracted configuration may be similar to, or identical to, the contracted configuration illustrated in FIGS. 3-4A with respect to file 100.

FIG. 6 shows an enlarged view of a circled area shown in FIG. 5. FIG. 7 shows an enlarged view of a circled area shown in FIG. 6.

FIG. 7 shows detail of a distal end of segment 501. The distal end of segment 501 includes circular face 707, tapered surface 703, and ribs 701. FIG. 7 also shows a proximal end of segment 503. The proximal end of segment 503 includes ribs 705. In operation, interlocking of a rib 701 and a rib 705 transfers rotational movement from segment 501 to segment 503 or vice versa. In operation, interlocking of a rib 701 and a rib 705 transfers rotational movement from segment 501 to segment 503 even when segment 503 deflects relative to segment 501.

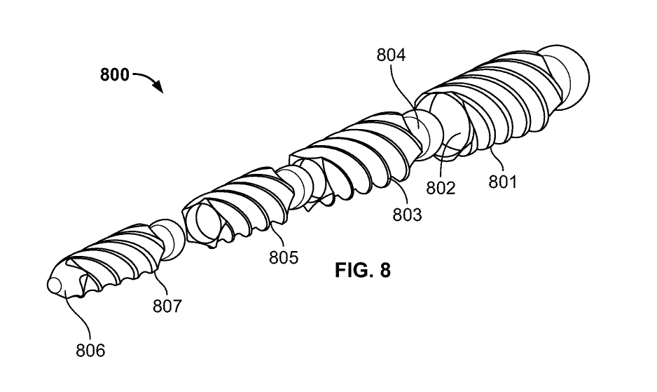

FIG. 8 shows illustrative apparatus 800. Apparatus 800 may be used to construct an articulating endodontic file. Apparatus 800 includes file segment 801, file segment 803, file segment 805 and file segment 807. One or more of the file segments may be fluted. Fluted file segments may have any suitable pitch or any suitable geometric parameters.

In some embodiments, apparatus 800 may include additional file segments (not shown). In some embodiments, apparatus 800 may include segment 807 and segment 805. In some embodiments, apparatus 800 may include segment 807, segment 805 and segment 803.

Apparatus 800 may include a handle (not shown). The handle may be coupled to a proximal end of apparatus 800. The handle may be used to drive one or more of segments 801, 803, 805 and 807 in rotation and/or translation.

Each of segment 801, segment 803, segment 805 and segment 807 may include a proximal circular head. Each of segment 801, segment 803, segment 805 and segment 807 may include a distal recess. The distal recess may be dome-shaped. The distal recess may define an articulating surface. The distal recess may be shaped to receive a circular head of an adjacent segment. Fitting the head into the recess may form a joint. The joint may be a ball-and-socket type joint. The joint may allow each segment to deflect relative to a longitudinal axis defined by an adjacent segment.

In some embodiments, a circular head of a file segment may fit into a distal recess of an adjacent segment by a snap-fit. The snap-fit may hold the two segments together without hindering motion of a circular head within the distal recess. In some embodiments, a circular head of a segment may be loosely positioned inside a distal recess of an adjacent segment without being coupled to the adjacent segment.

In some embodiments, a circular head, such as circular head 804, may extend from a distal end of a file segment. In such embodiments, a proximal end of the adjacent segment may include a recess configured to receive the circular head.

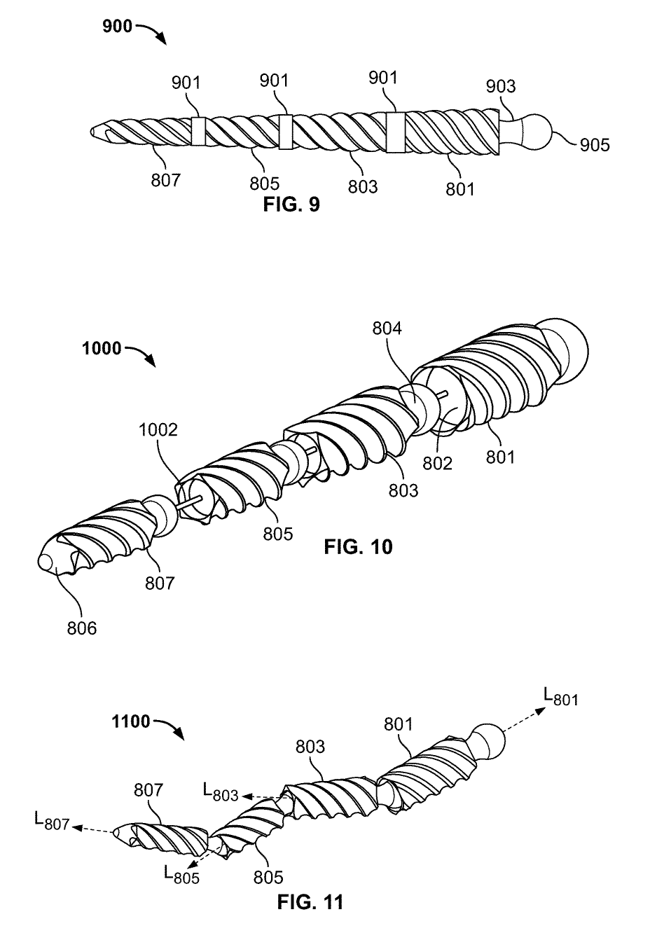

Some embodiments may include a spacer (shown below in FIG. 9) between a first segment and a second segment adjacent to the first segment. The spacer may couple a segment to an adjacent segment. The spacer may be made from flexible material, such as rubber. The spacer may fill a gap between two adjacent file segments.

The spacer may be fixedly or removably attached to a distal end of a first segment and/or a proximal end of a second segment. The spacer may be sufficiently rigid to transfer a rotational force from a first segment to a second segment. The second segment may be distal or proximal to the first segment.

In some embodiments, a central member (not shown) may extend through one or more of segments 801, 803, 805 and 807. The central member may be in addition to, or instead of, the spacer(s). In embodiments that include a central member, each segment housing the central member may include an inner lumen. The central member may extend through the inner lumens. An inner lumen may extend through an entire length of a file segment. An inner lumen may extend through less than the entire length of a file segment. In some embodiments, a segment may be crimped or welded onto a central member.

In some embodiments, an inner lumen may have a non-circular cross-section. The central member may have a cross-section corresponding to the cross-section of the inner lumen. Rotation of the central member passing through the inner lumen may transmit torque to a segment mounted on the central member. The torque may rotate the segment and cut tissue within the canal.

For example, the central member may include one or more sides, edges, vertices, protrusions and/or notches. The sides, edges, vertices, protrusions and/or notches may functionally engage the inner lumen of one or more of the segments through which the central member extends. An inner lumen of a segment may include a cross-sectional shape that corresponds to a cross-sectional shape of the central member. The cross-sectional shape may include features that allow the inner member to transfer rotational motion to the segment. The central member may drive, in rotation, the segments mounted on the central member.

FIG. 9 shows illustrative apparatus 900. Apparatus 900 may be used to form an articulating endodontic file. Apparatus 900 includes segments 801, 803, 805 and 807 (shown above in FIG. 8). Apparatus 900 includes spacers 901. Each spacer may function to space a distal end of a segment apart from a proximal end of an adjacent segment. The spacers may flexible. The spacers may prevent debris from accumulating between adjacent segments during a dental procedure. In some embodiments, at least a portion of a spacer may be compressed when a segment deflects relative to an adjacent segment.

FIG. 10 shows illustrative apparatus 1000. Apparatus 1000 may include one or more features of apparatus 800. Apparatus 1000 may be used to construct an articulating endodontic file. Apparatus 1000 includes central member 1002. Central member 1002 penetrates each of segments 1001, 1003, 1005 and 1007. FIG. 10 shows segments 1001, 1003, 1005 and 1007 mounted on central member 1002.

FIG. 11 shows illustrative operational view 1100 of apparatus 800 (shown above in FIG. 8). View 1100 shows segment 801, segment 803, segment 805 and segment 807 positioned in different directions with respect to each other. View 1100 shows each segment in apparatus 800 deflecting relative to a longitudinal axis of an adjacent segment.

View 1100 shows an exemplary positioning of circular heads (such as circular head 804 shown above in FIG. 8) within a recess (such as recess 802 shown above in FIG. 8). Apparatus 800 may include one or more spacers, such as spacer 901 (shown above in FIG. 9). A spacer may maintain a default (e.g., unstressed) positioning of a first segment relative to another segment.

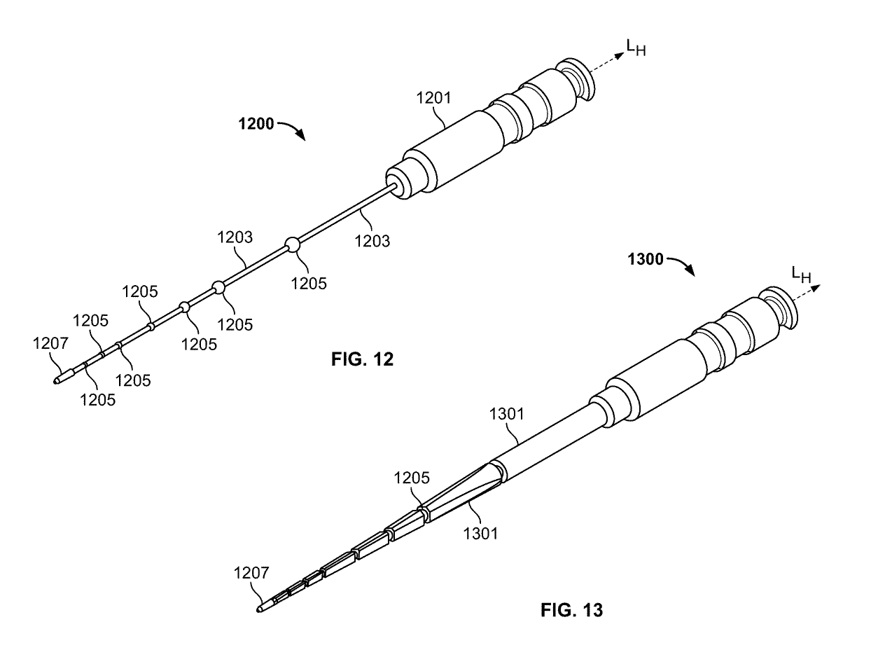

FIG. 12 shows illustrative apparatus 1200. Apparatus 1200 may be used to construct an articulating endodontic file. Illustrative apparatus 1200 includes handle 1201, central member 1203, spacers 1205 and tip 1207.

Spacers 1205 may be formed from any suitable material, such as rubber, metal or a metal alloy. Spacers 1205 may be flexible. Spacers 1205 may be rigid. In FIG. 12, spacers 1205 are circular. Spacers 1205 may be any suitable shape.

Central member 1203 may have any suitable cross-section. Central member 1203 may have a circular cross-section, a triangular cross-section, a square cross-section, or any other suitable cross-section, including one or more sides, edges or vertices.

Central member 1203 may be flexible. Central member 1203 may be substantially rigid. Central member 1203 may be partially rigid. Central member 1203 may have any suitable degree of flexibility.

FIG. 12 shows that handle 1201 defines a longitudinal axis L_{H} of apparatus 1200.

FIG. 13 shows illustrative articulating endodontic file 1300. In FIG. 13, file segments 1301 are shown mounted on central member 1203. Spacers 1205 are mounted on central member 1203 between segments 1301.

Segments 1301 may include an inner recess (not shown). The inner recess may be dome-shaped. The inner recess may receive at least a portion of circular spacer 1205. The inner recess may provide an articulating surface that allows a file segment 1301 to rotate or bend about a spacer 1205.

A segment 1301 may at least partially compress spacer 1205 when the segment 1301 deflects relative to an adjacent segment. In some embodiments, circular spacer 1205 may fit into an inner recess of segment 1301. In some embodiments, circular spacer 1205 may space a first segment apart from a second segment. In some embodiments, a spacer 1205 may not be compressed during deflection of file segments.

Segments 1301 may define any suitable cross-section. The cross-section may be circular, or may include any suitable number of sides and/or edges. Two sides may meet at or near an edge. The edge may form a vertex. Segments 1301 may be fluted. Segments 1301 may include one or more cutting edges. Segments 1301 may include one or more flutes, grooves, channels, ribs and/or ridges.

Segments 1301 may include an inner lumen. Central member 1203 may pass through the inner lumen of segments 1301. Central member 1203 may be fixedly or removably attached to the inner lumen of segments 1301. In some embodiments, central member 1203 may include one or more sides, edges or notches. Sides, edges or notches of central member 1203 may engage correspondingly shaped features of segments 1301.

A proximal end of central member 1203 may be affixed to handle 1201. A distal end of central member 1203 may be affixed to tip 1207. Central member 1203 may pass through one or more of segments 1301. A segment may be crimped on to a central member. Central member 1203 may be coupled or fixed to one or more of segments 1301. Central member 1203 may transfer rotational or translational force to segments 1301. Rotational or translational force may be applied to handle 1201 and may propagate, via central member 1203, to one or more of segments 1301.

A central member may not have a uniform diameter along its length. For example, central member 1203 may not include a uniform cross-section along its length.

Central member 1203 may have a circular cross-section. A file segment 1301 positioned along the length having the circular cross-section may include an inner lumen that also includes a circular cross-section. When central member 1203 is rotated, the file segment having the circular cross-section may not rotate. File segments adjacent to the segment having the circular cross-section may be configured to rotate when central member 1203 is rotated.

A segment 1301 may bend relative to an adjacent segment 1301. A segment 1301 may bend away from a longitudinal axis defined by the adjacent segment. A segment 1301 may bend away from longitudinal axis L_{H} defined by handle 1201. One or more of segments 1301 may be bendable and or flexible. One or more of segments 1301 may be bendable and or flexible when file is operating in a canal. For example, one or more of segments 1301 may be formed from an alloy having shape memory or superelastic properties.

Alloys having shape memory or superelastic properties may be configured to change shape in response to changes in temperature. The change in temperature may be triggered by being heated to body temperature. The change in temperature may be triggered by heated generated when the file is operating in the canal. Each file segment may be configured to respond to different trigger temperatures. Alloys having shape memory or superelastic properties may be configured to bend about a pivot point that is not a joint between two file segments. For example, a file segment may be configured to bend about a point on its own longitudinal axis.

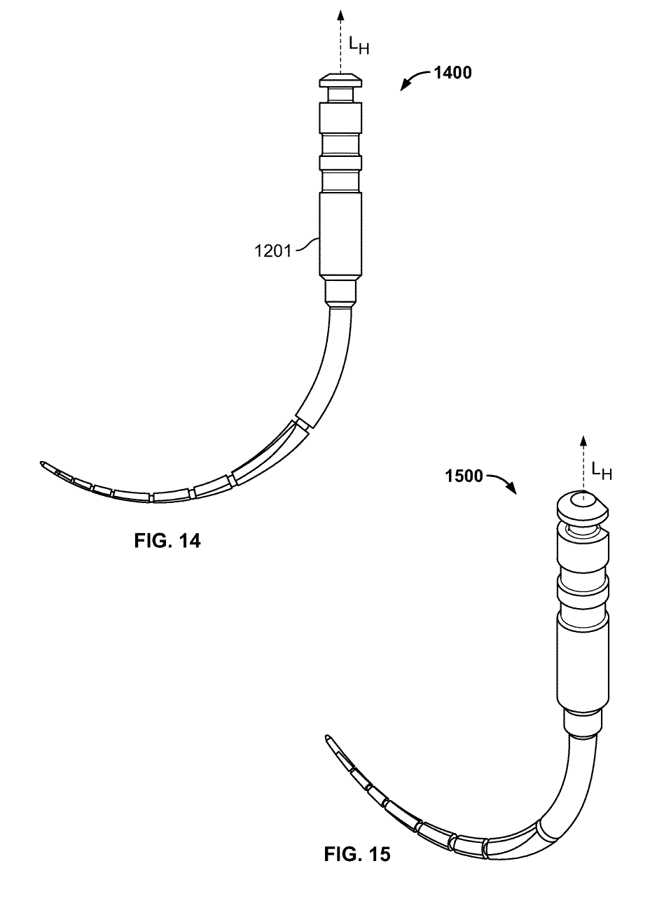

FIG. 14 shows an operational view 1400 of file 1200 (shown above in FIG. 12). View 1400 shows that segments 1301 may be flexible. In other embodiments, one or more of segments 1301 may be rigid. View 1400 shows that segments 1301 may deflect with respect to each other.

View 1400 shows segments 1301 bent away from longitudinal axis L_{H} of handle 1201. In operation, a segment may pivot or bend about a joint between two segments when following a natural curvature of a root canal within a tooth. In operation, a segment may pivot or bend about a point along its own longitudinal axis (e.g., the file segment is flexible). Tissue within the canal may have a lower density than the surrounding tooth tissue. In operation, segments 1301 may pivot or bend about a spacer when following a natural curvature of the canal. An articulating feature of file 1300, may reduce a likelihood that tip 1207 of file 1300 will cut tooth tissue outside a canal, enlarge an apical foramen or penetrate the apical foramen.

In some embodiments, a file segment may be configured to bend in response to a change in temperature. For example, a segment may be constructed from a shape memory alloy imbued with properties that increase flexibility of the segment at a pre-determined temperature or with a pre-determined range of temperatures.

FIG. 15 shows another operational view 1500 of file 1300 in a bent or deflected configuration.

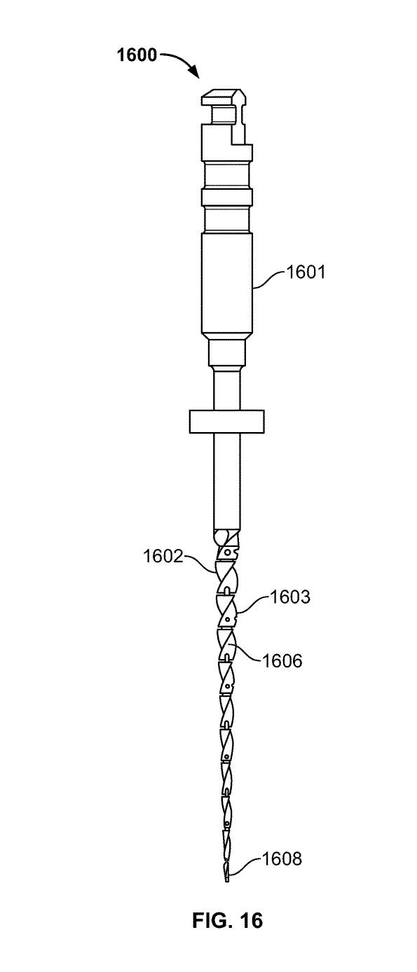

FIG. 16 shows illustrative articulating file 1600. File 1600 includes handle 1601, file segment 1602, file segment 1603, file segment 1606 and tip 1608.

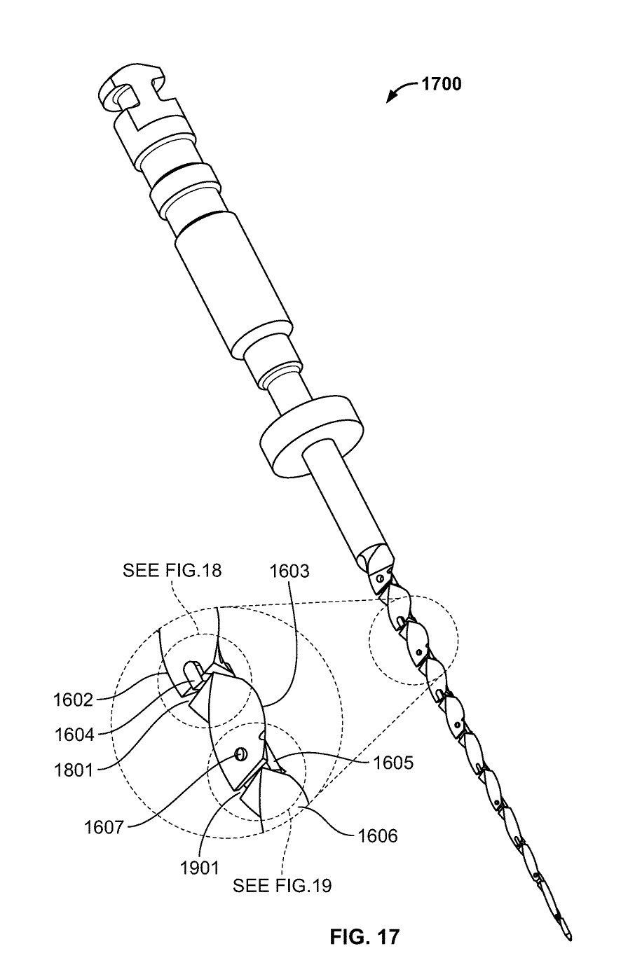

FIG. 17 shows perspective view 1700 of illustrative file 1600 (shown in FIG. 16). View 1700 shows that file segments 1602 and 1603 are linked to each other by a first joint. View 1700 shows that file segments 1603 and 1606 are linked to each other by a second joint.

View 1700 shows that segments 1602 and 1603 are each fluted. View 1700 also shows that, in the shown orientation, a flute may be contiguous from segment 1602 to a segment 1603.

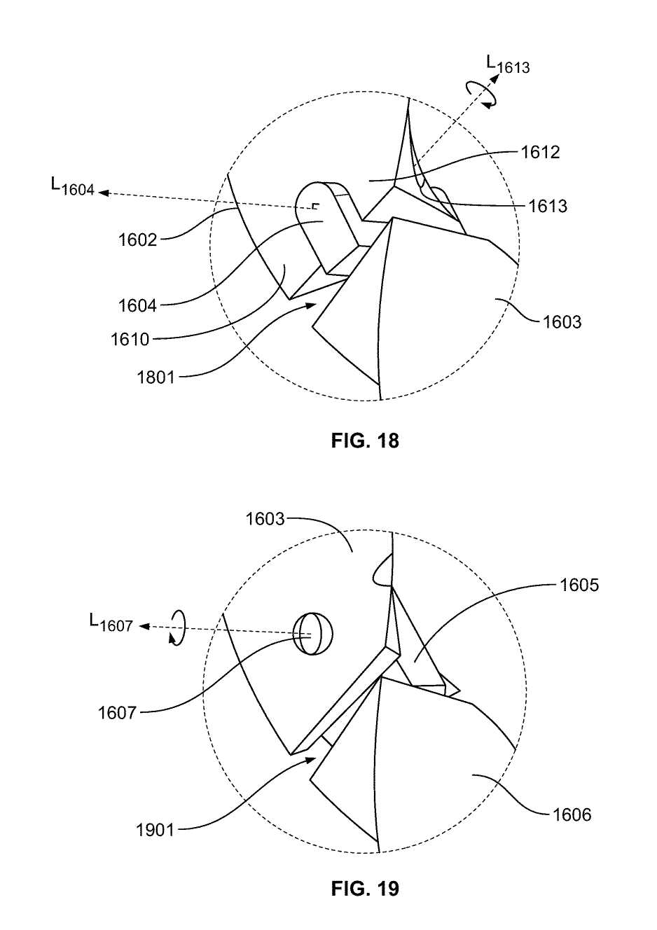

FIG. 18 shows a close-up view of a circled area (shown above in FIG. 17). FIG. 18 shows segment 1602 and segment 1603 and a joint between the segments. The joint allows segment 1602 to bend or deflect relative to adjacent segment 1603.

The first joint shown in FIG. 18 includes extension 1604 that fits into a distal “U” shaped cutout in segment 1602. A pin (not shown) may pass through first leg 1612 of the “U” shaped cutout, through extension 1604 and through second leg 1610 of the “U” shaped cutout. The pin (not shown) may be inserted into hole 1613. Gap 1801 allows segment 1602 to deflect relative to segment 1603 by rotating about axis L₁₆₁₃.

Compared to rotation about axis L₁₆₁₃, the joint shown in FIG. 18 may be relatively inflexible about axis L₁₆₀₄. In operation, the first joint may propagate rotational or translational force applied about axis L₁₆₀₄ to segment 1603.

Gap 1801 may space apart a distal end of a first segment such as 1602 from a proximal end of a second segment such as 1603. The second segment may be located adjacent, and distal to, the first segment. The first joint shown in FIG. 18 may enable the first segment to bend relative to the second segment. Gap 1801 may define a maximum angle of deflection between the first and second adjacent segments. For example, the first segment may bend or deflect relative to the second segment until the distal end of the first segment abuts the proximal end of the second segment.

In some embodiments, a distal end of the first segment may include a beveled edge. The proximal end of the second segment may also include a beveled edge. The beveled edges may provide a greater range of deflection between the first and second segment than non-beveled segments. The beveled edges may allow a proximal end of a distal segment to overlap a distal end of a proximal segment. In some embodiments, a distal end of a proximal segment may be spaced apart from the proximal end of a distal segment by a distance that is greater or less than gap 1801. Different size gaps may provide joints of differing ranges of deflection between segments. A single file may include different size gaps along a length of the file.

FIG. 19 shows that segment 1603 may be joined to segment 1606 by a second joint. The second joint includes extension 1605. Pin 1607 protrudes from extension 1605 and passes through segment 1603. In some embodiments, pin 1607 may pass through extension 1605. Pin 1607 and gap 1803 allow segment 1603 to deflect relative to segment 1606 by rotating about axis L₁₆₀₇.

Compared to rotation about axis L1607, the second joint shown in FIG. 19 may be relatively inflexible about an axis perpendicular to axis L₁₆₀₇. In operation, the first joint may propagate rotational or translational force applied about an axis perpendicular to axis L₁₆₀₇ to segment 1606.

Gap 1901 allows segment 1603 to deflect relative to segment 1606 by rotating about axis L₁₆₀₇. Gap 1901 may space apart a distal end of a first segment, such as segment 1603 from a proximal end of a second segment, such as segment 1606. The second segment may be located adjacent, and distal to, the first segment. The second joint may enable the first segment to bend or deflect relative to the second segment.

Gap 1901 may define a maximum angle of deflection between two adjacent segments. For example, the first segment may bend or deflect relative to the second segment until the distal end of the first segment abuts the proximal end of the second segment.

In some embodiments, a distal end of the first segment may include a beveled edge. The proximal end of the second segment may also include a beveled edge. The beveled edges may provide a greater range of deflection between the first and second segment than non-beveled segments. The beveled edges may allow a proximal end of a distal segment to overlap a distal end of a proximal segment. In some embodiments, a distal end of a proximal segment may be spaced apart from the proximal end of a distal segment by a distance that is greater or less than the gap 1901. Different size gaps may provide joints of differing ranges of deflection between segments. A single file may include different size gaps along a length of the file.

An articulating file, such as file 1600 (shown above in FIG. 16), may include any suitable number and/or combination of joints shown in FIG. 18 and/or FIG. 19.

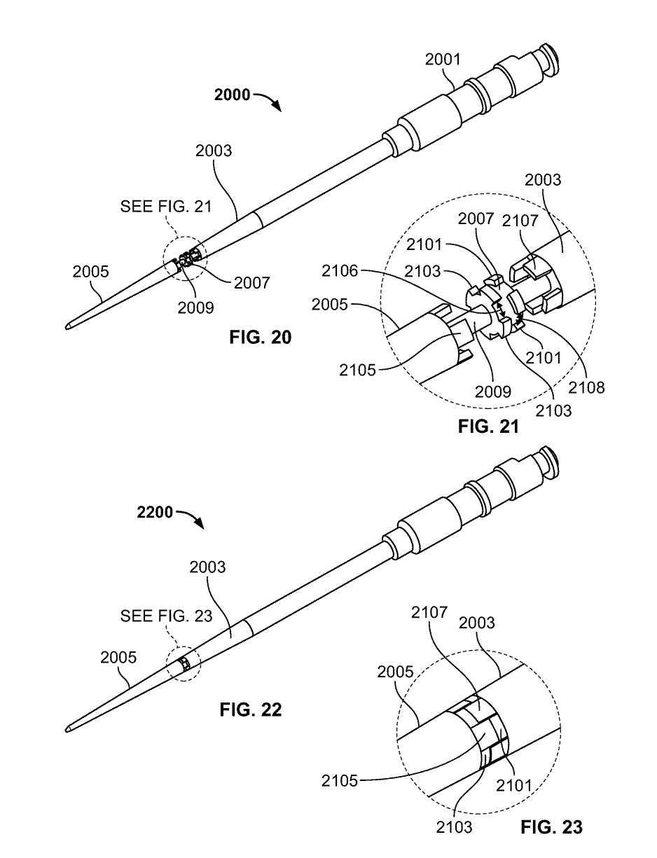

FIG. 20 shows illustrative components 2000 of an articulating file. Components 2000 includes handle 2001, segment 2003, segment 2005, spacer 2007 and central member 2009. FIG. 20 shows that components 2000 include two file segments. In other embodiments, an articulating endodontic file may include three, four, five or more file segments.

FIG. 20 shows components 2000 in an exploded view. The exploded view shows segment 2003, segment 2005 and spacer 2007 spaced apart from one another, with central member 2009 extending between segment 2003 and segment 2005.

One or both of segment 2003 and segment 2005 may be flexible. Segment 2003 and segment 2005 may each be flexible and deflect with respect to each other. Each segment of an endodontic file may have different flexibility properties. One or both of segment 2003 and segment 2005 may be rigid. Segment 2003 and segment 2005 may each have any suitable cross-section. Illustrative cross-sections may include a circular cross-section, rectangular cross-section, or any other suitable geometric cross-section. An illustrative cross-section may define one or more sides, edges or vertices where sides meet.

File segments may include any suitable features. For example, segment 2003 and/or segment 2005 may include one or more flutes, grooves, channels, ribs and/or ridges. Each segment may be configured to be positioned such that a flute runs contiguously from handle of an endodontic file to a tip of the file. Segment 2003 and/or segment 2005 may include one or more cutting edges. A flute may define the one or more cutting edges.

Central member 2009 may be attached to an inner portion of handle 2001. Central member 2009 may be attached to a distal tip of segment 2005. Central member 2009 may pass through a first lumen defined by segment 2003, a second lumen defined by segment 2005 and a third lumen defined by spacer 2007. Central member 2009 may be flexible. Central member 2009 may be substantially rigid. Central member 2009 may be partially rigid.

Central member 2009 may be fixedly or removably attached to inner lumens of segment 2003, segment 2005 and/or spacer 2007. In some of these embodiments, central member 2009 may include one or more sides, edges, vertices or notches. Features of central member 2009 may engage one or both of corresponding features of segment 2003, segment 2005 and/or spacer 2007. An exemplary corresponding feature may include a lumen shaped to receive vertex of central member 1203. Via engagement of the lumen and the vertex, central member 2009 may operationally transfer a rotational and/or translational force from segment 2003 to segment 2005. Central member 2009 may receive torque or other force from handle 2001. Handle 2001 may be may receive torque or other force from an electric drill or other tool.

Central member 2009 may be flexible. For example, central member 2009 may be formed from rubber or any other suitable material. An exemplary rubber used to form central member 2009 includes a thermoplastic vulcanizate (“TVP”) rubber such as Santoprene™.

FIG. 21 shows an enlarged view of a circled area shown in FIG. 20. FIG. 21 shows detail associated with spacer 2007. Spacer 2007 includes a plurality of distal protrusions 2103. The plurality of distal protrusions are distributed about an outer perimeter of spacer 2007. Spacer 2007 includes a plurality of proximal protrusions 2101. The plurality of proximal protrusions are distributed about an outer perimeter of spacer 2007. FIG. 21 shows the plurality of distal protrusions 2103 offset from the plurality of proximal protrusions 2101.

Spacer 2007 may include one or more notches. Each plurality of protrusions may define a plurality of notches between protrusions. For example, the plurality of proximal protrusions defines notch 2108. The plurality of distal protrusions defines notch 2106.

FIG. 21 also shows detail associated with a distal end of segment 2003 and detail associated with a proximal end of segment 2005. The distal end of segment 2003 includes fingers 2107. The proximal end of segment 2005 includes fingers 2105.

Proximal protrusions 2101 of spacer 2007 may be distributed such that when file 2000 is fully assembled, each of fingers 2107 are positioned within one of notches 2108 (between two adjacent protrusions 2101). When positioned with a notch, each of fingers 2107 may completely fill one of notches 2108. When positioned with a notch, each of fingers 2107 may exert pressure on two or more of proximal protrusions 2101. When positioned with a notch, each of fingers 2107 may extend along an outer surface of spacer 2007 until a distal face of the finger 2107 abuts a proximal face of protrusion 2103.

Proximal protrusions 2101 and distal protrusions 2013 may be flexible. Flexibility may include compressibility. For example, when segment 2005 deflects away from segment 2003, one or more of fingers 2105 may compress one or more of proximal protrusions 2101 or other portions of spacer 2007. When segment 2005 deflects away from segment 2003, one or more of fingers 2105 may slide out of notches 2106. When segment 2005 deflects away from segment 2003, one or more of fingers 2107 may slide out of notches 2108.

FIG. 22 shows an illustrative view 2200 of assembled components 2000 (shown in FIG. 20). FIG. 22 shows first segment 2003 and second segment 2005 abutting spacer 2007.

FIG. 23 shows an enlarged view of a circled area shown in FIG. 22. In FIG. 23, fingers 2107 completely fill notches 2108. In FIG. 23, fingers 2105 completely fill notches 2106.

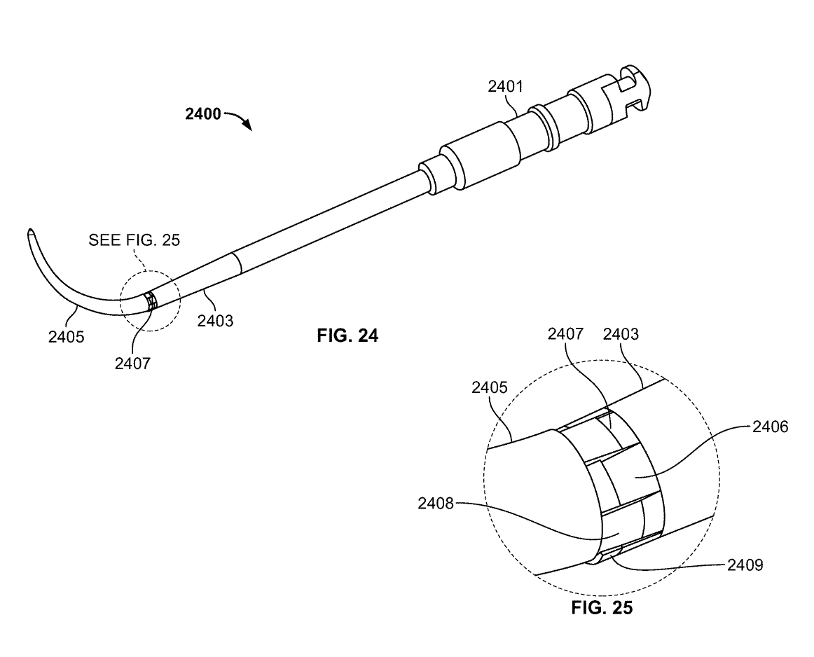

FIG. 24 shows illustrative articulating file 2400. Illustrative file 2400 includes handle 2401, segment 2403, segment 2405, and spacer 2407. Illustrative file 2400 may include a central member as illustrated in FIG. 20. File 2400 may include one or more features of components 2000 (shown in FIG. 20).

Segment 2403 and segment 2405 may have one or more features in common with segment 2003 and segment 2005 (shown in FIG. 20). Spacer 2407 may have one or more features in common with spacer 2007 (shown in FIG. 20).

Segment 2405 may be bendable. Segment 2403 may be bendable. Segment 2405 may be formed from an alloy such as nickel-titanium that has shape memory and/or superelastic properties. Segment 2405 may include shape memory and/or superelastic properties different from the shape memory and/or superelastic properties of segment 2403. In operation, articulating file segments 2405 and 2403 may allow file 2400 to more closely follow a root canal's natural curvature. Segment 2405 may bend relative to segment 2403. Segment 2405 may bend by compressing at least a portion of spacer 2407.

FIG. 25 shows an enlarged view of a circled area shown in FIG. 24. FIG. 25 shows that when, in operation, two file segments deflect with respect to each other, the position of fingers 2406 and fingers 2408 may shift. FIG. 25 also shows that when, in operation, segments deflect with respect to each other, the segments may exert forces on spacer 2407. The forces may change a shape of spacer 2407. The forces may shift a position of a feature (such as a protrusion) of spacer 2407. For example, FIG. 25 shows that when segment 2405 deflects with respect to segment 2403, protrusion 2409 is not flush with an outer surface of segment 2405.

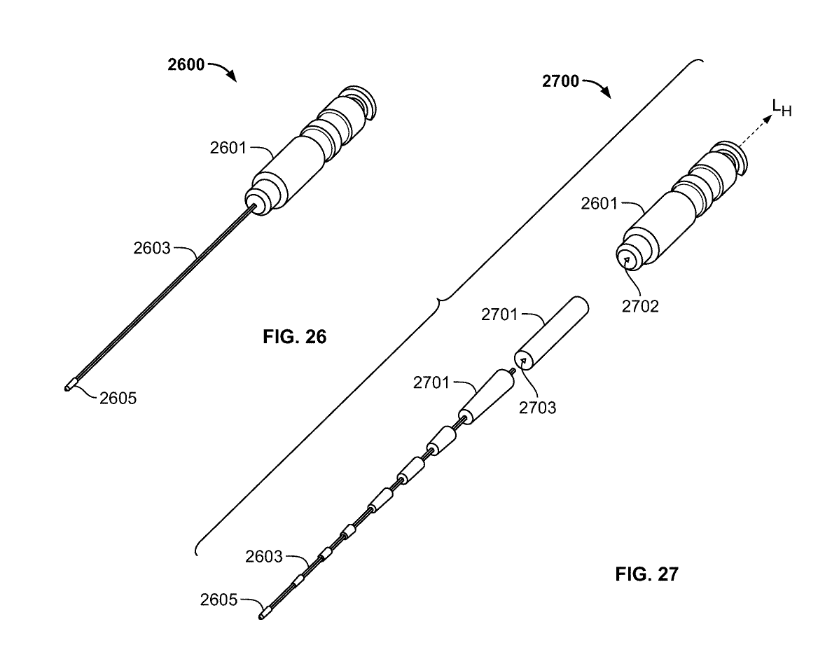

FIG. 26 shows illustrative components 2600 of an articulating endodontic file. Components 2600 include handle 2601, central member 2603 and tip 2605.

Central member 2603 may have any suitable cross-section. For example, central member 2603 may have a circular cross-section, a triangular cross-section, a square cross-section, or any other suitable geometric cross-section. A cross-section may define one or more sides, edges and/or vertices.

Central member 2603 may be flexible. Central member 2603 may be substantially rigid. Central member 2603 may be partially rigid. FIG. 26 shows central member 2603 having a triangular cross-section.

FIG. 27 shows an exploded view of illustrative articulating file 2700. File 2700 may include one or more features of components 2600 (shown in FIG. 26) and/or articulating file 1300 (shown in FIG. 13). For example, file 2700 may include spacers, such as spacers 1205 (shown in FIG. 13) between illustrative file segments.

FIG. 27 shows file segments 2701 mounted on central member 2603 (shown in FIG. 26). FIG. 27 shows that central member 2603 has a triangular cross-section. Segments 2701 may be slidably mounted on central member 2603.

One or more of segments 2701 may be bendable. One or more of segments 2701 may be rigid. For example, all of segments 2701 may be bendable or all of segments 2701 may be rigid. One or more of segments 2701 may be formed from a shape memory alloy.

FIG. 27 shows that segments 2701 have a triangular shaped inner lumen 2703. Triangular shaped inner lumen 2703 may be sized to receive triangular shaped central member 2603. When a segment 2701 is mounted on central member 2603, and a force is applied to central member 2603, vertices of triangular shaped central member 2603 may engage vertices of inner lumen. Engagement of the vertices may transfer force from central member 2603 to segment 2701.

Segments 2701 may have any suitable cross-section. The cross-section may be circular or may include any suitable number of sides, edges and/or vertices. Segments 2701 may include one or more cutting edges. Segments 2701 may be fluted. Segments 2701 may include one or more sides, flutes, grooves, channels, ribs and/or ridges.

Segments 2701 may include an inner lumen, such as inner lumen 2703. Central member 2603 may pass through inner lumen 2703 of segments 2701. Central member 2603 may be fixedly or removably attached to inner lumen 2703.

A proximal end of central member 2603 may be attached to handle 2601. The proximal end of central member 2603 may be fitted into recess 2702. FIG. 27 shows that recess 2702 has a triangular shape.

A distal end of central member 2603 may be attached to tip 2605. Central member 2603 may be coupled or fixed to segments 2701. Central member 2603 may transfer rotational or translational force to segments 2701. The rotational or translational force may be applied to handle 2601. For example, handle 2601 may be rotated by a motor.

One or more of segments 2701 may bend relative to an adjacent segment 2701. A segment 2701 may bend away from a longitudinal axis L_{H} defined by handle 2601.

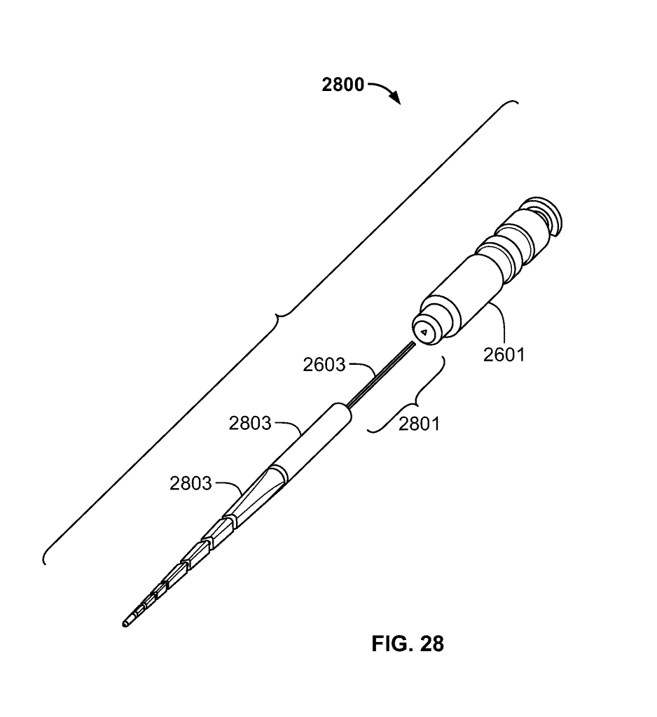

FIG. 28 shows illustrative apparatus 2800. Apparatus 2800 includes segments 2803 mounted onto central member 2603 (shown in FIG. 26). FIG. 28 shows a portion 2801 of central member 2603. Portion 2801 of central member 2603 may be a portion of central member 2603 that is fixed to an inner portion of handle 2601. FIG. 28 shows that portion 2801 has a triangular cross-section. Other portions (not shown) of central member 2603 may have different shaped cross-sections. FIG. 28 also shows that segments 2803 may each have different size and/or shaped cross-sections.

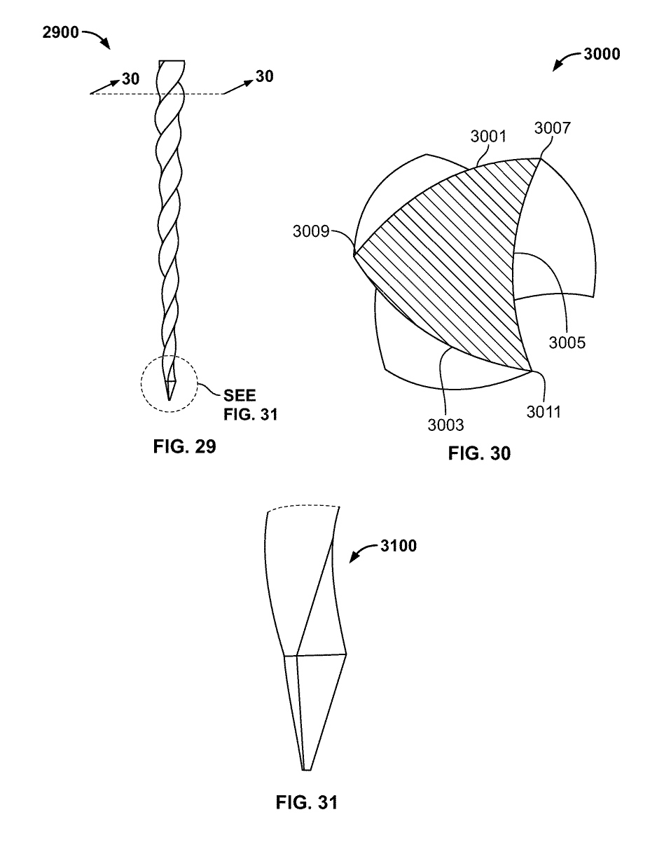

FIGS. 29-41 show illustrative endodontic file designs and associated cross-sectional views. Cross-sectional views may be described with respect to an area of the cross-section relative to a reference circular perimeter that encircles the cross-sectional view or profile.

One or more articulating files and/or file segments disclosed herein may have geometric properties similar to, or identical to those shown in FIGS. 29-41.

FIG. 29 shows illustrative working length 2900 of an endodontic file. Working length 2900 includes helical twists. The helical twists correspond to flutes and associated cutting edges of working length 2900.

FIG. 30 shows cross-section 3000 of the file shown in FIG. 29. Cross-section 3000 is taken along cut lines 30-30 shown in FIG. 29. Cross-section 3000 includes concave side 3005. Cross-section 3000 includes convex side 3001 and 3003. Cross-section 3000 includes three cutting edges—3007, 3009 and 3011.

A cutting edge may define a positive or negative rake angle. A rake angle is formed by an axis perpendicular to a surface of material to be removed (e.g., tooth tissue) and a cutting edge. A cutting edge may define a positive rake angle when a file is rotated in a first direction (about a central longitudinal axis). The cutting edge may define a negative rake angle when the file is rotated in a second direction (about a central longitudinal axis). The second direction may be opposite to the first direction.

For example, when cross-section 3000 is rotated in a clockwise direction, cutting edge 3011 exhibits a positive rake angle. When cross-section 3000 is rotated in a counterclockwise direction, cutting edge 3011 exhibits a negative rake angle.

FIG. 31 shows a close up view of file tip 3100 (also shown in FIG. 29). During shaping of a canal, a file tip may enlarge the canal and guide the file through the canal. These tip functionalities may be accomplished by balancing geometric features of the tip. Illustrative geometric features may include a rake angle of the tip's cutting edge, a radius of the tip's cutting edge and the proximity of a flute end to the tip end.

In some embodiments, a tip may be completely landed to form a complete circle by defining a conical shape with a small radius (also known as a “complete tip”). The tip may be rounded or circularly shaped. A tip that forms a complete circle may be unable to cut tissue or may have poor cutting abilities. An articulating file may have any suitable tip geometry. For example, a tip may have a conventional tip size in the range of 06 to 100.

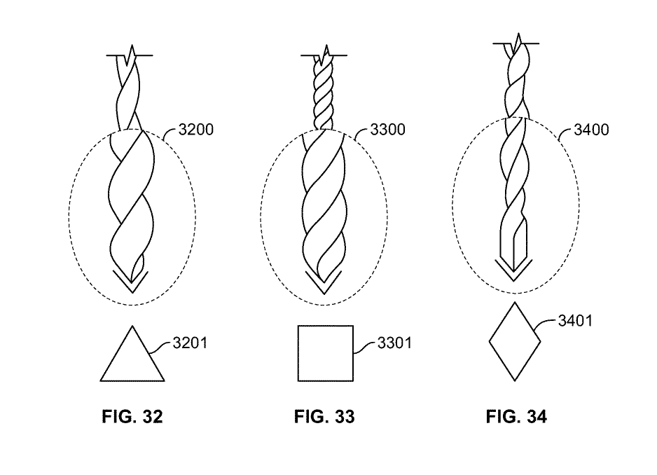

FIG. 32 shows length 3200 of an illustrative endodontic file. Length 3200 has a three-sided triangular cross-section 3201. A segment of an articulating file may have a triangular cross-section.

Generally, a flute may define two edges. One cutting edge and one trailing edge, respectively. A triangular cross-section file, as shown in FIG. 32, has three flutes and six theoretical edges. However, due to the triangular shape of the cross-section, the trailing edge of flute 1 is also the cutting edge of the flute 2, the trailing edge of the flute 2 is also the cutting edge of the flute 3, and the trailing edge of flute 3 is also the cutting edge of flute 1. Therefore, a triangular cross-section typically includes three cutting edges.

FIG. 33 shows length 3300 of an illustrative endodontic file. Length 3300 has a four-sided square-shaped cross-section 3301. A segment of an articulating file may have a square-shaped cross-section.

FIG. 34 shows length 3400 of an illustrative endodontic file. Length 3400 has a four-sided diamond-shaped cross-section 3401. A segment of an articulating file may have a diamond-shaped cross-section.

FIG. 35 shows length 3500 of an illustrative endodontic file. Length 3500 has asymmetric cross-section 3501. A segment of an articulating file may have an asymmetrically-shaped cross-section such as cross-section 3501.

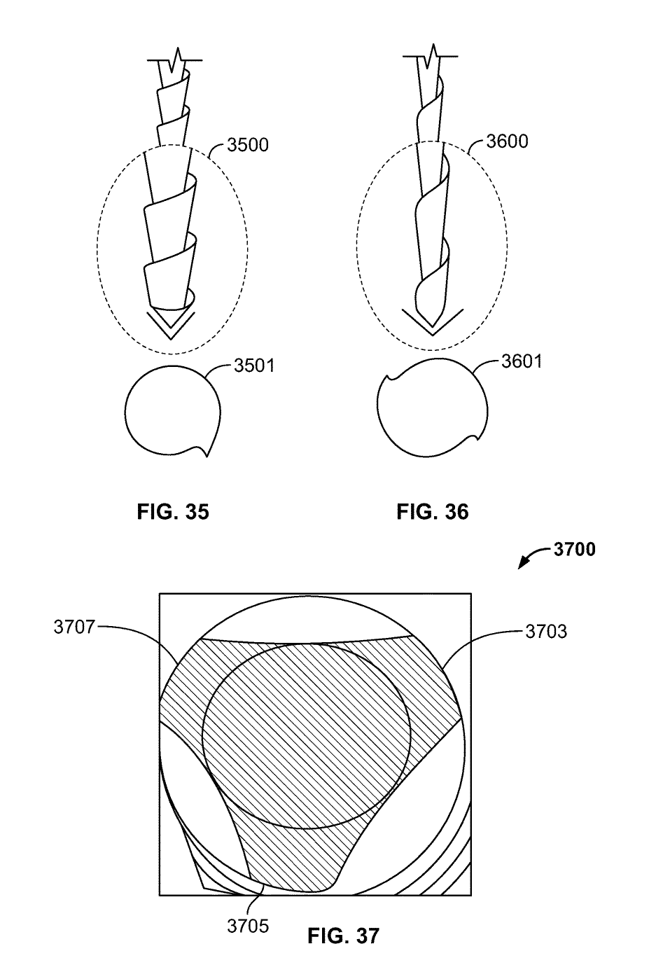

FIG. 36 shows length 3600 of an illustrative endodontic file. Length 3600 has asymmetric cross-section 3601. A segment of an articulating file may include an asymmetrically-shaped cross-section such as cross-section 3601.

FIG. 37 shows illustrative cross-section 3700. Cross-section 3700 has a six-sided triangular shape. Cross-section 3700 includes land surfaces 3703, 3705 and 3707. A land surface (“land”) may be a surface extending along an outer face of the working length. The land may space a helical twist of a flute apart from an adjacent helical twist. For example, in embodiments where a file includes two flutes, the land of the file may be larger than the land of an endodontic file having three or more flutes.

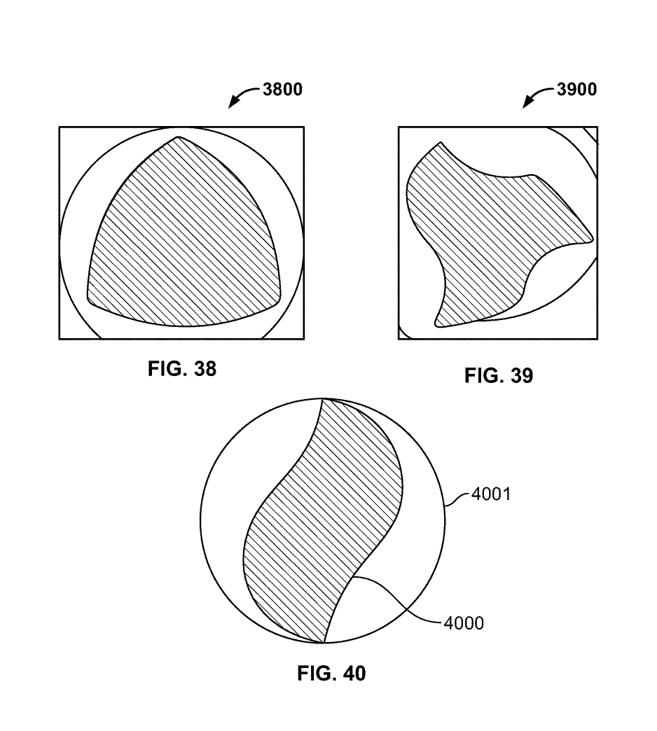

FIG. 38 shows illustrative triangular cross-section 3800. Triangular cross-section 3800 includes three convex sides. One or more file segments of an articulating endodontic file may include triangular cross-section 3800.

FIG. 39 shows illustrative asymmetrical cross-section 3900. Cross-section 3900 includes curved sides. Curved sides may enhance debris removal capability of an endodontic file. One or more file segments of an articulating endodontic file may include one or more features of triangular cross-section 3900.

FIG. 40 shows illustrative cross-section 4000. FIG. 40 shows that a cross-section may be defined based on reference circular perimeter 4001. Reference circular perimeter 4001 circumscribes cross-section 4000. A cross-section may be defined based on a percentage of an area occupied by the cross-section within the reference circular perimeter.

An articulating endodontic file may be defined by a plurality of geometric properties. Exemplary geometric properties include a central longitudinal axis, an inner lumen, fluted areas, a plurality of cross-sections, a pitch, a taper, a helix angle, a removable area coefficient (“RAC”), and a number of helical twists of the flute along a working length of the file. Any of the geometric properties detailed herein may be combined with any other geometric properties detailed herein to create a file with target performance parameters.

For example, RAC may be defined as:
RAC=Circumscribed Area/Material Cross-section Area/No. of Cutting Edges
   Thus, multiple cutting edges typically reduce the file's RAC value.

In some embodiments, one or more of the geometric properties of the file may be constant along a length of a file segment. In some embodiments, one or more of the geometric properties of the file may vary along the length of the file segment. Each segment of an articulating file may have geometric properties different from other segments in the articulating file.

For example, one or more of the size and/or shape of the file's cross-section, the pitch, the taper, the helix angle, the RAC value and/or the location of a central axis of the grove may vary along a length of the file and/or a file segment.

In some embodiments, a cutting edge of a file segment may have variable positive or negative rake angle along its length. In some embodiments, the cutting edge of a file segment may have both positive and negative rake angles along its length.

A cutting edge of a file segment, according to certain embodiments, may have a constant or variable helix angle. The helix angle may be an angle that the cutting edge forms with a longitudinal axis of the segment. A longitudinal axis may include an axis extending along an outer perimeter of a file segment's length.

A file segment may define one or more cross-sections along its length. The cross section(s) may be symmetric cross-sections, asymmetric cross-sections, or symmetric and asymmetric cross-sections. The cross-sections may have any suitable geometric properties.

A file segment, according to some embodiments, may include material properties such as one or both of shape memory and superelasticity. Each file segment in an articulating file may have its own unique shape memory and superelasticity properties. For example, a file segment closer to a file tip may have a first set of shape memory and superelasticity properties. A file segment closer to a file handle may have a second set of shape memory and superelasticity properties. A file segment positioned in a mid-section of an articulating file may have a third set of shape memory and superelasticity properties.

Geometric properties of the file, according to certain embodiments, described herein may be properties that the file has at room temperature. Geometric properties of a file may be configured to change in response to heat, cooling, stress or other stimuli.

A file segment may be machined from a tube, an elongated rod, a flat bar, a round bar, a square bar, a triangular bar, or any other suitable shaped blank. The machining of the file segment may include one or both of heat treatment and variable heat treatment.

The blank may be solid or hollow. The blank may be a tube, a cylinder, a flat sheet of material or may have conical shape. The blank may have a circular cross-section, a square cross-section, a triangular cross-section, a rectangular cross-section, an oval cross-section, a diamond cross-section, a hexagon cross-section, or any other suitable cross-section.

The cross-section of the blank may taper along the length of the blank. The cross-section of the blank may be constant along the length of the blank.

The cross-section of the blank may vary along the length of the blank. For example, a blank may be selected, or machined, such that at predetermined intervals along the length of the blank the cross-section of the blank is shifted in one direction (e.g., to the left), relative to a central axis of the blank. The cross-section of the blank may be shifted back in the opposite direction (e.g., to the right) at the next interval. The shifting may be offset by any suitable angle or any suitable distance. In some embodiments, the blank may be shifted by a fraction of a millimeter, by 1 mm, or by any suitable distance.

An articulating endodontic file may include two or more cutting edges. Cutting edges may be formed by flutes in a file segment. Flutes by be formed in a file segment by twisting any of the blanks described above. A blank may be twisted around a mandrel or around its center to create the flutes. For example, a file segment may be manufactured using one or more features of the heat treat methods and apparatus that are shown and described in U.S. Pat. No. 6,783,438, which is hereby incorporated herein by reference in its entirety. In some embodiments, the blank may be heat treated, twisted, and then heat treated again.

A file segment may be manufactured using electrical discharge machining (EDM) techniques on any of the blanks described above. For example, the file may be manufactured using one or more features of the EDM methods and apparatus that are shown and described in U.S. Pat. No. 7,207,111, which is hereby incorporated herein by reference in its entirety.

A file segment may be manufactured using electrochemical machining (ECM) techniques on any of the blanks described above. A file segment may be manufactured by laser cutting any of the blanks described above. A file segment may be manufactured using 3D printing techniques.

Thus, apparatus and methods for an articulating endodontic file have been provided. Persons skilled in the art will appreciate that the present disclosure can be practiced by other than the described examples, which are presented for purposes of illustration rather than of limitation. The present disclosure is limited only by the claims that follow.

What is claimed is:

1. An articulating endodontic file comprising:
a first segment defining a longitudinal axis and comprising a first rib;
a second segment joined to the first segment and comprising a second rib;
wherein, in operation:
the first rib engages the second rib and transmits force from the first segment to the second segment; and
the second segment is configured to deflect relative to the longitudinal axis while receiving the force.

2. The articulating endodontic file of claim 1 further comprising a central member that joins the first segment to the second segment.

3. The articulating endodontic file of claim 1 wherein:
the first rib is one of a plurality of first ribs protruding from a distal end of the first segment; and
the second rib is one of a plurality of second ribs protruding from a proximal end of the second segment.

4. The articulating endodontic file of claim 3, wherein the plurality of first ribs are spaced about the distal end of the first segment such that each second rib in the plurality of second ribs is configured to fit between two adjacent first ribs in the plurality of first ribs.

5. The articulating endodontic file of claim 1 wherein the force is torque.

6. The articulating endodontic file of claim 3 wherein the first segment is configured to transmit the force to the second segment when at least one rib in the plurality of first ribs interlocks with at least one rib in the plurality of second ribs.

7. The articulating endodontic file of claim 3 wherein, in operation, when the second segment deflects relative to the longitudinal axis of the first segment, at least one rib in the plurality of second ribs interlocks with at least one rib in the plurality of first ribs.

8. The articulating endodontic file of claim 1 further comprising a flexible joint between a distal end of the first segment and a proximal end of the second segment.

9. The articulating endodontic file of claim 8, wherein, in operation, the second segment is configured to deflect relative to the longitudinal axis by bending about the flexible joint.

10. The articulating endodontic file of claim 9, wherein, in operation, the flexible joint provides the second segment a range of motion between 0 and 90 degrees in any direction relative to the longitudinal axis of the first segment.

11. The articulating endodontic file of claim 1 wherein:
the first segment comprises at least one cutting edge; and
the second segment comprises at least one cutting edge.

12. The articulating endodontic file of claim 11 wherein the second cutting edge is contiguous with the first cutting edge when the longitudinal axis of the first segment is aligned with a longitudinal axis of the second segment.

13. An articulating endodontic file comprising:
a first segment comprising a head; and
a second segment comprising a recess that is configured to receive the head.

14. The articulating endodontic file of claim 13 further comprising a spacer between the first segment and the second segment.

15. The articulating endodontic file of claim 13 wherein, the head is configured to snap-fit into the recess.

16. The articulating endodontic file of claim 13 further comprising a central member that joins the first segment to the second segment.

17. The articulating endodontic file of claim 16 wherein:
the first segment comprises a first inner lumen;
the second segment comprises a second inner lumen; and
the central member passes through the first inner lumen and passes through the second inner lumen.

18. The articulating endodontic file of claim 17 wherein:
the first inner lumen extends through the head; and
the second inner lumen extends through the recess.

19. The articulating endodontic file of claim 17 wherein:
the first inner lumen comprises a non-circular cross-section; and
the central member comprises a cross-section that is configured to engage the non-circular cross-section of the first inner lumen.

20. The articulating endodontic file of claim 19 wherein, in operation, the central member transmits force to the first segment.

21. The articulating endodontic file of claim 20 wherein the force is torque.

22. The articulating endodontic file of claim 13 wherein:
the first segment is fluted; and
the second segment is fluted.

23. An articulating endodontic file comprising:
a central member;
a first segment mounted on the central member; and
a second segment mounted on the central member.

24. The articulating endodontic file of claim 23:
the first segment comprising a first inner lumen;
the second segment comprising a second inner lumen; and
the central member comprising a non-circular cross-section that is configured to:
pass through the first inner lumen and pass through the second inner lumen; and
transmit force to the first segment and to the second segment.

25. The articulating endodontic file of claim 23 further comprising a spacer positioned between the first segment and the second segment.

26. The articulating endodontic file of claim 25:
the first segment comprising a distal recess configured to receive a proximal end of the spacer; and
the second segment comprising a proximal recess configured to receive a distal end of the spacer.

27. The articulating endodontic file of claim 23:
the first segment further comprising a first finger extending from a distal end of the first segment;
the second segment further comprising a second finger extending from a proximal end of the second segment.

28. The articulating endodontic file of claim 27 further comprising a spacer comprising at least two protrusions defining at least one notch such that the first finger or the second finger is configured to be seated in the notch.

29. An articulating endodontic file comprising:
a first file segment;
a second file segment;
a joint linking the first file segment to the second file segment;
wherein, in operation, the second file segment rotates about the joint in a plane perpendicular to a longitudinal axis of the first file segment.

30. The articulating endodontic file of claim 29 wherein, the joint is a first joint and the plane is a first plane, the articulating endodontic file further comprising:
a third file segment; and
a second joint linking the second file segment to the third file segment;
wherein, in operation, the third file segment rotates about the second joint in a second plane that is:
perpendicular to the longitudinal axis of the first file segment; and
perpendicular to the first plane.

31. The articulating endodontic file of claim 29, the joint comprising:
a proximal extension affixed to a proximal end of the second file segment;
a distal cutout in a distal end of the first file segment, the distal cutout configured to receive the proximal extension and defining a first leg and a second leg; and
a pin that is configured to pass through the first leg, through the extension and through the second leg, thereby pinning the second file segment to the proximal extension.

32. The articulating endodontic file of claim 30, the second joint comprising:
a proximal extension affixed to a proximal end of the third file segment;
a pin affixed to the proximal extension and is configured to pass through the second file segment thereby pinning the third file segment to the proximal extension.
